(19) United States
(12) Patent Application Publication
(10) Pub. No.: US 2026/0232471 A1
(43) Pub. Date: Aug. 13, 2026

(54) LOW FRICTION STENT DELIVERY SYSTEM

(71) Applicant: BOSTON SCIENTIFIC SCIMED, INC., Maple Grove, MN (US)

(72) Inventors: Gary Gilmartin, Foxford (IE); Daniel Tuck, Oughterard (IE); Keith O'Reilly, Kilcolgan (IE); Shane O'Keeffe, Tuam (IE); Fionn Stapleton, Oranmore (IE); Celine Glynn, Galway (IE); Amanda Matashu, Sligo (IE); Thomas Martin Keating, Tuam (IE); John Thomas O'Driscoll, Galway (IE); David Giles, Galway (IE)

(73) Assignee: BOSTON SCIENTIFIC SCIMED, INC., Maple Grove, MN (US)

(21) Appl. No.: 19/465,495

(22) Filed: Jan. 30, 2026

Related U.S. Application Data
(60) Provisional application No. 63/753,588, filed on Feb. 4, 2025.

(51) Int. Cl. A61F 2/966; A61F 2/95; A61M 25/00

(52) U.S. Cl. CPC A61F 2/966; A61F 2/9517; A61M 2025/0004

(57) ABSTRACT

This disclosure addresses inaccuracies in stent deployment due to the frictional forces experienced by a co-axial delivery system within an endoscope. The disclosed stent delivery systems incorporate a pull thread assembly to retract a stent retention tube to release a self-expanding stent. The disclosed systems and method may include additional components to maintain the integrity of the pull thread assembly. The stent delivery system may include a floating tubular bumper and/or eyelets to contain the pull threads of the pull thread assembly along an exterior of an inner shaft of the stent delivery system. These configurations reduce friction between the components of the stent delivery system and the working channel of an endoscope, enabling more precise stent deployment by preventing inadvertent movement and premature release.

### CROSS REFERENCE TO RELATED APPLICATIONS

The present application claims the benefit of US Provisional Patent Application Serial No. 63/753,588, filed February 4, 2025, the disclosure of which is incorporated herein by reference.

### TECHNICAL FIELD

The present disclosure generally relates to deployment of self-expanding stents in co-axial stent delivery systems. More specifically, the present disclosure is related to improving accuracy of stent placement and deployment through the incorporation of a pull thread assembly to reduce frictional deployment forces of the stent delivery system.

### BACKGROUND

In a co-axial stent deployment system, a catheter shaft, which may be inserted through the working channel of an endoscope in some instances, may include an outer shaft slidably disposed over an inner shaft. To deploy a self-expanding stent therefrom, the outer shaft is withdrawn proximally relative to the inner shaft to expose the stent from the distal end of the outer shaft, thereby allowing the stent to radially expand from a radially constrained configuration within the outer shaft.

However, significant friction exists when attempting to withdraw the outer tube, such as between the outer shaft and the working channel of the endoscope, which may cause inadvertent movement of the catheter shaft, causing the stent to release prematurely and/or off-target. Thus, to reduce inaccuracies in stent deployment, a need to reduce and manage the frictional forces experienced by the stent delivery system, such as frictional forces between the stent delivery system and the working channel of an endoscope, is desired.

### BRIEF SUMMARY

This disclosure provides design, material, manufacturing method, and use alternatives for medical devices, including stent delivery devices.

In an example, a stent delivery system includes a stent and a catheter shaft. The catheter shaft includes an inner shaft having proximal and distal end regions, and a stent retention tube slidably disposed over the distal end region of the inner shaft. The stent retention tube is configured to hold the stent in a constrained position and deploy the stent when retracted proximally. The system includes a pull thread assembly with one or more pull threads extending along the inner shaft, where the pull threads are fixedly secured to the stent retention tube and extend proximally along the inner shaft. The pull threads are configured to transmit a proximal retraction force to retract the stent retention tube.

Alternatively or additionally to any of the examples above, the catheter shaft includes a floating tubular bumper circumferentially surrounding the proximal end region of the inner shaft.

Alternatively or additionally to any of the examples above, the one or more pull threads extend between the inner shaft and the floating tubular bumper.

Alternatively or additionally to any of the examples above, the stent retention tube is axially movable relative to the floating tubular bumper.

Alternatively or additionally to any of the examples above, the stent retention tube is axially movable between a distal delivery position where the stent is restrained and a proximal deployment position where the stent is deployed.

Alternatively or additionally to any of the examples above, in the distal position, a distal end of the floating tubular bumper is spaced proximally away from the proximal end of the stent retention tube, and in the proximal position the proximal end of the stent retention tube is moved toward the distal end of the floating tubular bumper.

Alternatively or additionally to any of the examples above, in the proximal position, the distal end of the floating tubular bumper abuts the proximal end of the stent retention tube.

Alternatively or additionally to any of the examples above, in the proximal position, the stent retention tube overlaps the floating tubular bumper.

Alternatively or additionally to any of the examples above, the floating tubular bumper does not move axially when the stent retention tube is moved from the distal position to the proximal position.

Alternatively or additionally to any of the examples above, the system includes a transition tube extending proximally from the proximal end of the stent retention tube to a distal end region of the floating tubular bumper.

Alternatively or additionally to any of the examples above, the system includes a handle assembly at the proximal end of the inner shaft, where the floating tubular bumper is free from direct attachment to the handle assembly.

Alternatively or additionally to any of the examples above, the one or more pull threads extend proximal of a proximal end of the floating tubular bumper to the handle assembly.

Alternatively or additionally to any of the examples above, the pull thread assembly includes a plurality of guide eyelets positioned longitudinally along the inner shaft, where the pull threads are threaded through the guide eyelets.

Alternatively or additionally to any of the examples above, the stent retention tube has an outer diameter and the floating tubular bumper has an outer diameter, where the outer diameter of the floating tubular bumper is less than the outer diameter of the stent retention tube.

Alternatively or additionally to any of the examples above, the stent retention tube has an outer diameter and the floating tubular bumper has an inner diameter, where the inner diameter of the floating tubular bumper is more than the outer diameter of the stent retention tube.

In another example, a stent delivery system includes a stent and a catheter shaft. The catheter shaft includes an inner shaft having proximal and distal end regions, and a stent retention tube slidably disposed over the distal end region of the inner shaft. The stent retention tube is configured to hold the stent in a constrained position and deploy the stent when retracted proximally. The system includes a pull thread assembly with one or more pull threads extending along the inner shaft, where the pull threads are fixedly secured to the stent retention tube and extend proximally along the inner shaft. The pull threads are configured to transmit a proximal retraction force to retract the stent retention tube.

Alternatively or additionally to any of the examples above, the catheter shaft includes a floating tubular bumper circumferentially surrounding the proximal end region of the inner shaft.

Alternatively or additionally to any of the examples above, the one or more pull threads extend between the inner shaft and the floating tubular bumper.

Alternatively or additionally to any of the examples above, the stent retention tube is axially movable relative to the floating tubular bumper.

Alternatively or additionally to any of the examples above, the stent retention tube is axially movable between a distal delivery position where the stent is restrained and a proximal deployment position where the stent is deployed.

Alternatively or additionally to any of the examples above, in the distal position, a distal end of the floating tubular bumper is spaced proximally away from the proximal end of the stent retention tube, and in the proximal position the proximal end of the stent retention tube is moved toward the distal end of the floating tubular bumper.

Alternatively or additionally to any of the examples above, in the proximal position, the distal end of the floating tubular bumper abuts the proximal end of the stent retention tube.

Alternatively or additionally to any of the examples above, in the proximal position, the stent retention tube overlaps the floating tubular bumper.

Alternatively or additionally to any of the examples above, the floating tubular bumper does not move axially when the stent retention tube is moved from the distal position to the proximal position.

Alternatively or additionally to any of the examples above, the system includes a transition tube extending proximally from the proximal end of the stent retention tube to a distal end region of the floating tubular bumper.

Alternatively or additionally to any of the examples above, the system includes a handle assembly at the proximal end of the inner shaft, where the floating tubular bumper is free from direct attachment to the handle assembly.

Alternatively or additionally to any of the examples above, the one or more pull threads extend proximal of a proximal end of the floating tubular bumper to the handle assembly.

Alternatively or additionally to any of the examples above, the pull thread assembly includes a plurality of guide eyelets positioned longitudinally along the inner shaft, where the pull threads are threaded through the guide eyelets.

Alternatively or additionally to any of the examples above, the stent retention tube has an outer diameter and the floating tubular bumper has an outer diameter, where the outer diameter of the floating tubular bumper is less than the outer diameter of the stent retention tube.

Alternatively or additionally to any of the examples above, the stent retention tube has an outer diameter and the floating tubular bumper has an inner diameter, where the inner diameter of the floating tubular bumper is more than the outer diameter of the stent retention tube.

In another example, a stent device delivery system includes a stent and a catheter shaft. The catheter shaft includes a handle assembly and an inner shaft extending distally from the handle assembly having proximal and distal end regions. A stent retention tube is slidably disposed over the distal end region of the inner shaft with the proximal end spaced apart distally of the handle assembly. The stent retention tube is configured to hold the stent in a constrained position and deploy the stent when retracted proximally. The system includes a pull thread assembly with one or more pull threads fixedly secured to the stent retention tube, extending from a proximal end of the stent retention tube along an exterior of the proximal end region of the inner shaft to the handle assembly. The pull threads are configured to transmit a proximal retraction force to retract the stent retention tube upon actuation of the handle assembly.

Alternatively or additionally to any of the examples above, the catheter shaft includes a floating tubular bumper circumferentially surrounding the proximal end region of the inner shaft.

Alternatively or additionally to any of the examples above, the one or more pull threads extend between the inner shaft and the floating tubular bumper.

Alternatively or additionally to any of the examples above, the pull thread assembly includes a plurality of guide eyelets positioned longitudinally along the proximal end region of the inner shaft, where the pull threads are threaded through the guide eyelets.

In another example, a method of deploying a stent includes advancing a stent delivery system to a treatment site, where the system includes a self-expanding stent, a catheter shaft with an inner shaft having proximal and distal end regions, and a stent retention tube slidably disposed over the inner shaft and axially moveable between a distal delivery position where the stent is in a constrained position and a proximal deployment position where the stent is deployed. The system includes a pull thread assembly with one or more pull threads fixedly secured to the stent retention tube and extending proximally along the inner shaft. The method includes retracting the stent retention tube proximally relative to the stent from the distal delivery position to the proximal deployment position by transmitting a proximal retraction force through the pull threads, causing the stent to self-expand at the target site.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE SEVERAL VIEWS OF THE DRAWINGS

The disclosure may be more completely understood in consideration of the following detailed description in connection with the accompanying drawings, in which:

FIG. 1 is a side view of an exemplary stent delivery system.

FIG. 2 is an enlarged view of a portion of the exemplary stent delivery system of FIG. 1.

FIG. 3 is a cross-sectional view of a portion of the exemplary stent delivery system of FIG. 1 taken along line 3-3.

FIG. 4A is a side view of an exemplary stent delivery system prior to deployment of the stent.

FIG. 4B is a side view of the exemplary stent delivery system of FIG. 4A after deployment of the stent in accordance with another embodiment of the subject matter disclosed herein.

FIG. 4C is a side view of the exemplary stent delivery system of FIG. 4A after deployment of the stent in accordance with an embodiment of the subject matter disclosed herein.

FIG. 5A is a side view of another exemplary stent delivery system prior to deployment of the stent in accordance with an embodiment of the subject matter disclosed herein.

FIG. 5B is a side view of the exemplary stent delivery system of FIG. 5A after deployment of the stent in accordance with an embodiment of the subject matter disclosed herein.

FIG. 6 illustrates a portion of a stent delivery system in accordance with another embodiment of the subject matter disclosed herein.

FIG. 7 illustrates a stent delivery system in accordance with another embodiment of the subject matter disclosed herein.

FIG. 8 is a block method diagram in accordance with the subject matter disclosed herein.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

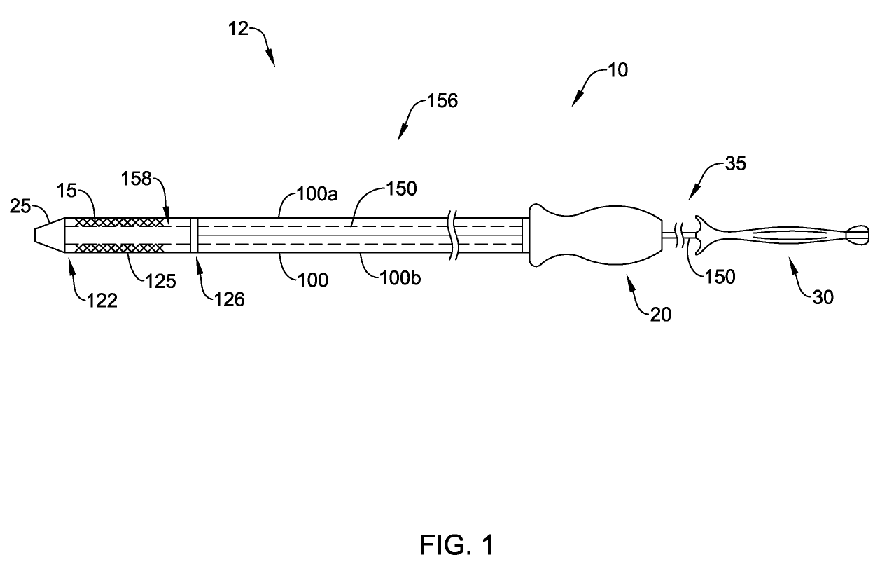

FIG. 1 shows a side view of a stent delivery system 10. Stent delivery system includes a catheter shaft 12 to deliver and deploy a self-expanding stent 15 in a body lumen of a patient. The catheter shaft 12 may include a stent retention tube 125, an inner shaft 150, and a pull thread assembly 100 extending proximally from the stent retention tube 125, such as extending proximally from the proximal end of the stent retention tube 125, along the proximal end region 156 of the inner shaft 150. The pull thread assembly 100 may include one or more, or a plurality of pull threads 100a, 100b, etc. secured to the proxima1 end 126 of stent retention tube 125 and extending proximally therefrom. The one or more, or plurality of pull threads 100a, 100b, etc. of the pull thread assembly 100 may be exposed to an exterior of the catheter shaft 12 along a portion of the length, along a majority of the length, and/or along substantially the entire length of the catheter shaft 12 between the stent 15 and the handle assembly 35, in some instances. The one or more, or plurality of pull threads 100a, 100b, etc. may extend along an exterior of the inner shaft 150 to a handle assembly 35 provided at a proximal end of the stent delivery system 10.

The distal end of the inner shaft 150 is secured to a distal tip 25, with the stent 15 positioned proximal thereof. Prior to deployment, the stent 15 surrounds a distal end region 158 of the inner shaft 150 and is positioned within a lumen of the stent retention tube 125 such that stent 15 is compressably constrained by the stent retention tube 125 at the distal end region 158 of the inner shaft 150. The distal end 122 of stent retention tube 125 may contact and/or abut the distal tip 25 with the stent retention tube 125 slidably disposed over the stent 15 and the distal end region 158 of the inner shaft 150. The stent retention tube 125 may be withdrawn proximally relative to the inner shaft (and thus the distal tip 25) to uncover the stent 15 constrained on the distal end region 158 of the inner shaft 150 in order to deploy the stent 15. The stent retention tube 125 may have a length, measured from the distal end 122 of the stent retention tube 125 to the proximal end 126 of the stent retention tub 125 sufficient to extend over the full length of the stent 15. However, the length of the stent retention tube 125 may be substantially less than the length of the catheter shaft 12, and substantially less than the length of the inner shaft 150 between the stent 15 and the handle assembly 35.

To operate, the stent delivery system 10 includes a handle assembly 35 which includes a first, distal handle 20 and a second, proximal handle 30. The first, distal handle 20 may be actuated (e.g., moved proximally) relative to the second, proximal handle 30 to deploy the stent 15. The proximal handle 30 may be connected to the proximal end of the inner shaft 150, while the distal handle 20 may be connected to the pull thread assembly 100. Once the stent 15 has been advanced within the catheter shaft to the target site within a body lumen, the user, typically a physician or medical technician, may actuate the distal handle 20 proximally toward the proximal handle 30, which in turn withdraws the stent retention tube 125 proximally, to deploy the stent 15. When the distal handle 20 is actuated proximally, the pull thread assembly 100 is also proximally retracted to the same degree or amount. Consequently, the individual pull threads 100a, 100b, etc. of the pull thread assembly 100 transmit a proximal retraction force to the stent retention tube 125, in turn causing the stent retention tube 125 to retract proximally relative to the stent 15, exposing and releasing the stent 15 from its radially constrained configuration to a radially expanded, deployed configuration placed at the target site.

In some conventional stent delivery systems, an outer tube extends longitudinally from the distal tip 25 to the distal handle 20 and surrounds the inner shaft 150. In such a configuration, stent deployment is achieved by proximal retraction of the outer tube relative to the inner shaft 150. However, significant friction may exist along the length of the outer tube and the working channel of an endoscope though which the stent delivery system extends through. The frictional forces may cause inadvertent movement of the outer tub and/or the inner shaft, causing the stent to release prematurely, off-target, or otherwise compromise operation of the stent delivery system.

In the stent delivery system 10, by replacing a full-length outer tube with the shorter stent retention tube 125 and pull thread assembly 100, the surface contact area, and subsequently the friction, between longitudinally moving components of the catheter shaft 12 and the working channel of an endoscope is reduced. By incorporating the pull thread assembly design, the stent delivery system 10 achieves improved deployment accuracy through better friction management.

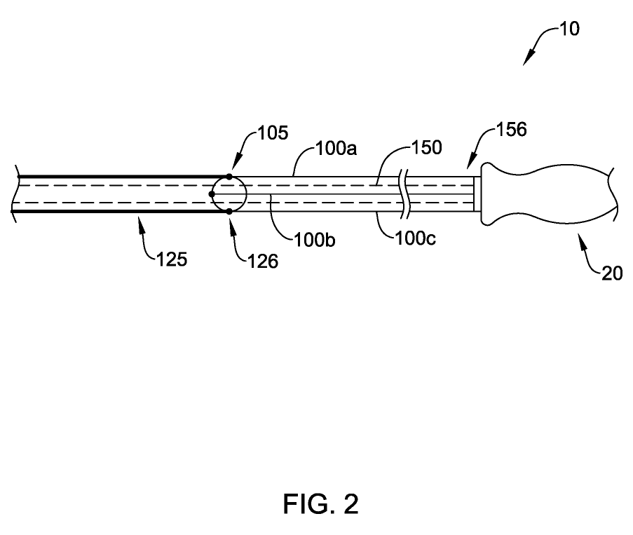

As shown in FIG. 2, the pull thread assembly 100 may include any desired quantity of pull threads. For example, the pull thread assembly 100 may include a first pull thread 100a, a second pull thread 100b, and a third pull thread 100c, in some instances. The pull threads 100a, 100b, 100c may be secured to the stent retention tube 125 via connection points 105 on the stent retention tube 125. The connection points 105 may be symmetrically or asymmetrically spaced around the circumference of the stent retention tube 125. The connection points 105 may be located proximate the proximal end 126 of the stent retention tube 125, or at another location if desired. Therefrom, the pull thread assembly 100 extends along the exterior of the proximal end region 156 of the inner shaft 150 (e.g., the portion of the inner shaft 150 extending proximal of the stent retention tube 125, to be connected to the distal handle 20. The proximal ends of the pull threads 100a, 100b, 100c may be fixedly secured to the distal handle 20 such that a tensile force is exerted on the pull threads 100a, 100b, 100c via proximal actuation of the distal handle 20 in order to proximally retract the stent retention tube 125.

The pull threads 100a, 100b, 100c may be manufactured from nylon, metal, or other materials with sufficient tensile strength and resistance to withstand retraction forces when pulled proximally. The chosen material may provide a degree of kink resistance such that the pull threads 100a, 100b, 100c remain sufficiently taut and maintain a straight path as the pull threads, 100a, 100b, 100c extend from the connection point 105 on the stent retention tube 125 along an outer surface of the proximal end region 156 of the inner shaft 150. In some embodiments, a degree of resistance to bending or coiling also ensures the pull thread assembly 100 maintains a smooth profile, and the individual pull threads 100a, 100b, 100c stay in their desired configuration. Additional consideration to material selection of pull threads 100a, 100b, 100c may include the ability of the material to withstand repeated proximal retraction force without stretching, twisting, entanglement, sagging, draping, or other deformation to the pull threads 100a, 100b, 100c.

In the present disclosure, the pull thread assembly 100 is illustrated with the individual pull threads 100a, 100b, 100c running parallel to the central longitudinal axis of the inner shaft 150 on an exterior of the inner shaft 150. In other embodiments, individual pull threads 100a, 100b, 100c of the pull thread assembly 100 may extend helically around the inner shaft 150 or be otherwise arranged along the inner shaft 150.

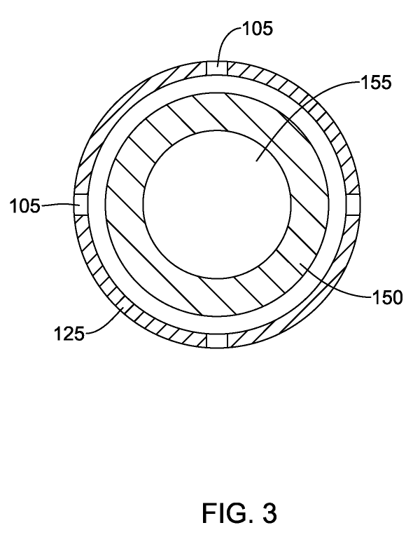

FIG. 3 shows a cross-sectional view of stent retention tube 125. In some embodiments, and as illustrated, the connection points 105 are openings extending through the wall of the stent retention tube 125. In this example, the pull threads 100a, 100b, 100c may be attached to the stent retention tube 125, using the openings 105 as pass-through points in which the pull threads 100a, 100b, 100 extend through the openings 105 and are tied or otherwise secured thereto. For example, the pull threads 100a, 100b, 100c, may then be knotted to securely attach the pull threads 100a, 100b, 100c, to the stent retention tube 125. In other embodiments, the pull threads 100a, 100b, 100c, can be secured to the outer surface or the inner surface of the stent retention tube 125 by adhesive or heat sealing. In other embodiments, a fitted sleeve or collar with openings may be placed over the outer diameter of the stent retention tube 125, or within the lumen of the stent retention tube 125, which may serve as connection points 105 for the pull threads 100a, 100b, 100c. Other secure attachment mechanisms of the pull threads 100a, 100b, 100c are also contemplated.

As can be seen in FIG. 3, the stent retention tube 125 may have a larger inner diameter than the outer diameter of the inner shaft 150 such that the stent retention tube 125 may be slidably disposed over the inner shaft 150. As such, the stent may be radially constrained between the stent retention tube 125 and the inner shaft 150 prior to deployment. Additionally, the inner shaft 150 may include a lumen 155 to receive a guidewire in some instances. The guidewire may be used to provide a path for the advancement of the stent delivery system 10, position the stent 15 at the target site, and otherwise help in accurate deployment.

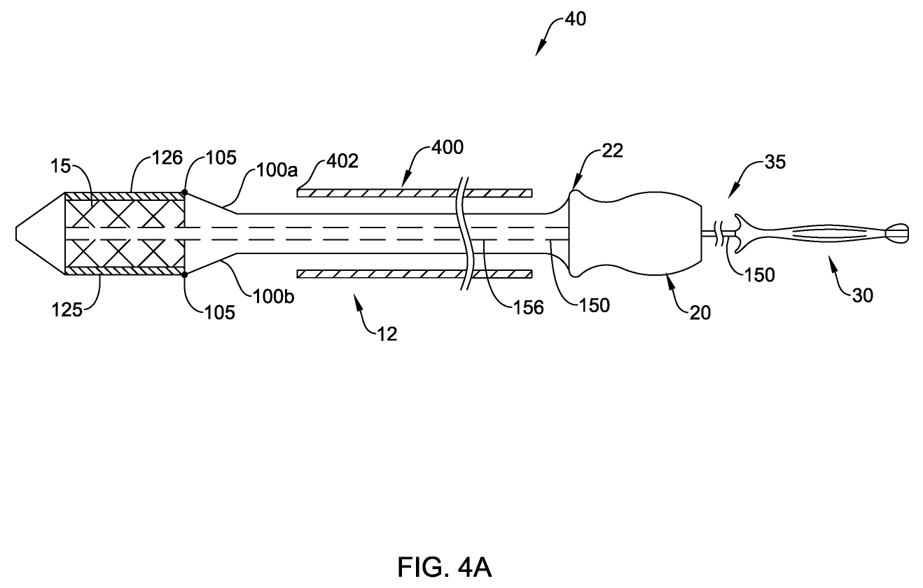

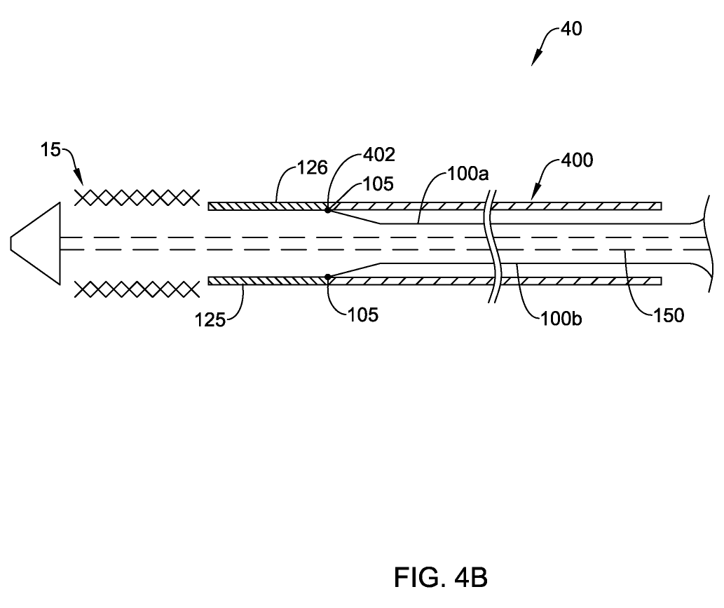

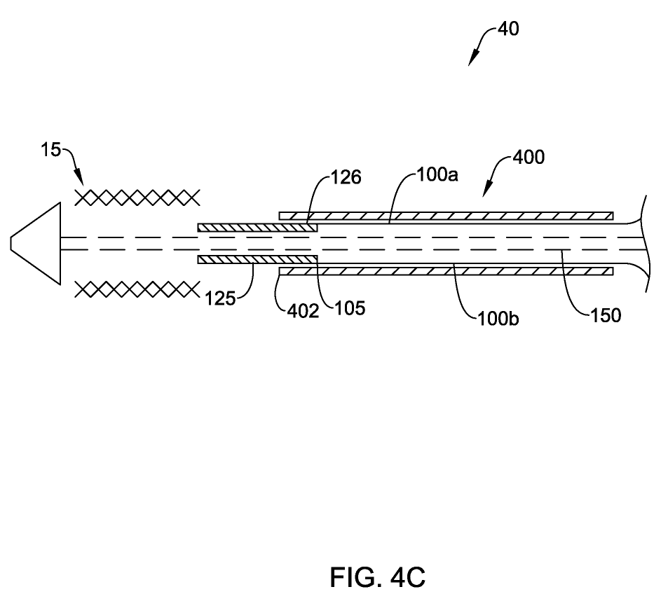

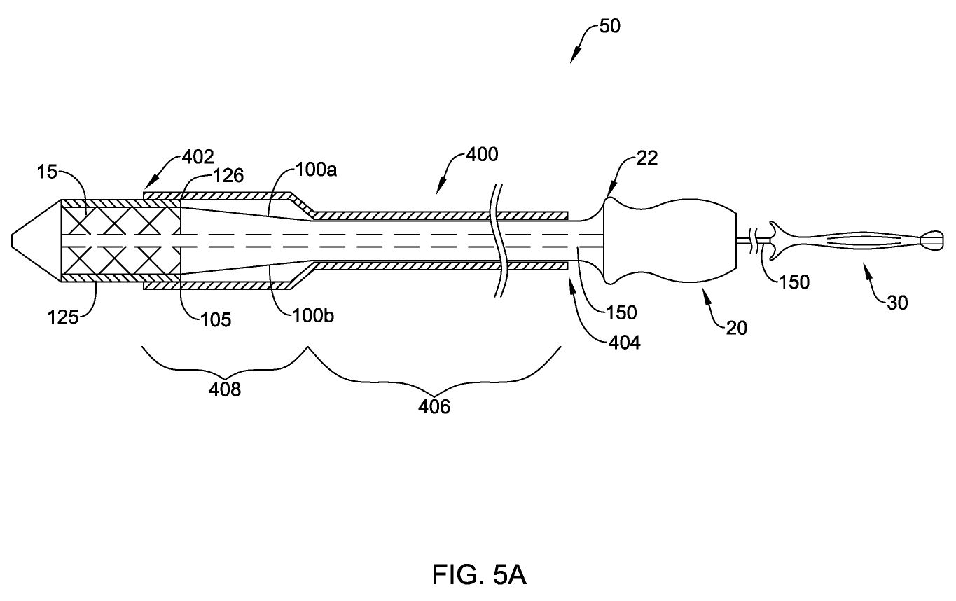

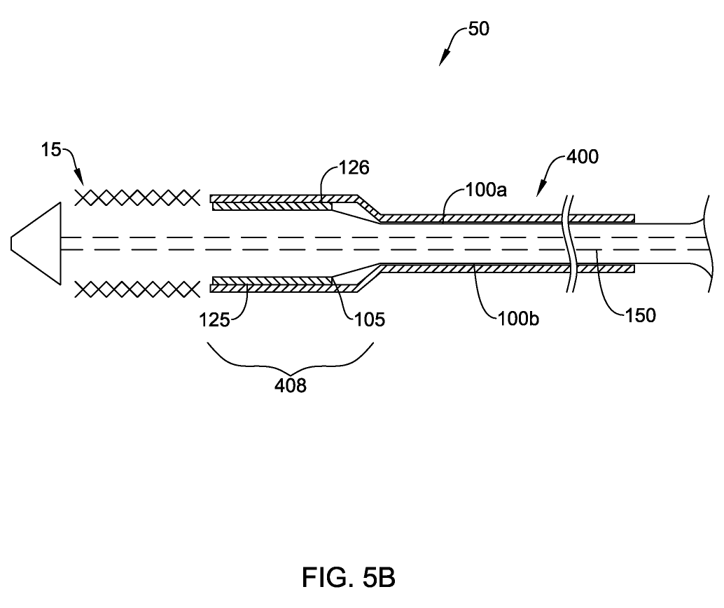

In some embodiments, the catheter shaft 12 may include a floating tubular bumper, as illustrated in FIGS. 4A-4C, and FIGS. 5A-B.

FIG. 4A shows a stent delivery system 40 in which the catheter shaft 12 includes a floating tubular bumper 400 surrounding the inner shaft 150 proximal of the stent 15. FIG. 4A illustrates the stent delivery system 40 with the stent retention tube 125 in a distal, delivery position, where the stent retention tube 125 surrounds and radially constrains the stent 15.

The floating tubular bumper 400 may circumferentially surround the proximal end region 156 of the inner shaft 150 with the pull threads 100a, 100b extending proximally from the stent retention tube 125 between the inner shaft 150 and the floating tubular bumper 400 to the distal handle 20. When positioned thereover, the floating tubular bumper 400 keeps the pull threads 100a, 100b in close proximity to the exterior of the inner shaft 150, ensuring the integrity of the pull thread assembly 100 and consistent alignment of the pull threads 100a, 100b during deployment, thereby maintaining the reduced friction benefits of the pull thread assembly design of the stent delivery system 40.

In some examples, the floating tubular bumper 400 may be configured as a singular tube having a length of approximately 50 cm or more, 70 cm or more, or 100 cm or more, for example. In some instances, the floating tubular bumper 400 may be configured as a singular tube having a length of approximately 250 cm or less, 200 cm or less, 150 cm or less, or 100 cm or less, for example. In some instances, the floating tubular bumper 400 may be configured as a singular tube having a length in the range of 50 cm to 250 cm, in the range of 50 cm to 200 cm, in the range of 50 cm to 150 cm, in the range of 100 cm to 250 cm, in the range of 100 cm to 200 cm, or in the range of 100 cm to 150 cm, for example. The floating tubular bumper 400 may have a length less than a distance from the proximal end of the stent 15 to the distal end of the distal handle 20, such as a length that is 95% or less than, or 90% or less than the distance from the proximal end of the stent 15 to the distal end of the distal handle 20. In other examples, the floating tubular bumper 400 may be configured as a series of separate floating tubular bumper segments extending along the pull threads 100a, 100b of the pull thread assembly 100. In some instances, the floating tubular bumper 400 may be a plurality of telescoping or nesting tubular segments configured to move axially relative to one another in an overlapping fashion. The multiple tubular bumpers 400 may be placed anywhere along the proximal end region 156 of the inner shaft 150 and may be uniform or vary in length and/or diameter.

The diameter of the floating tubular bumper 400 is sized to accommodate the pull threads 100a, 100b and proximal retraction of the stent retention tube 125 into the deployment position. In some instances, the outer diameter of the floating tubular bumper 400 may be 21 French (7 mm) or less, 18 French (6 mm) or less, 15 French (5 mm) or less, 12 French (4 mm) or less, or 6 French (2 mm) or less. In some instances, the outer diameter of the floating tubular bumper 400 may be in the range of 5 French (1.67 mm) to 21 French (7 mm), in the range of 6 French (2 mm) to 15 French (5 mm), or in the range of 6 French (2 mm) to 12 French (4 mm), for example. The distal end of the floating tubular bumper 400 may be positioned proximal of the proximal end of the stent retention tube 125 in the delivery configuration, with a gap between the distal end of the floating tubular bumper 400 and the proximal end of the stent retention tube 125. Thus, the stent retention tube 125 may be spaced away from and not directly secured to the floating tubular bumper 400 such that the stent retention tube 125 may be withdrawn proximally relative to the floating tubular bumper 400 independent of movement of the floating tubular bumper 400. Thus, the pull thread assembly 100, including the pull threads 100a, 100b, may be actuated proximally independent of and/or without proximal movement of the flotation tubular bumper 400 to withdraw the stent retention tube 125 proximally relative to the sent 15 to deploy the stent 15. In the initial distal, delivery position, as illustrated in FIG. 4A, the distal end 402 of the floating tubular bumper 400 is spaced proximally away from the proximal end 126 of the stent retention tube 125.

In some examples, the stent delivery system 40 may include a handle assembly 35 including the distal handle 20 and the proximal handle 30. In some examples, the floating tubular bumper 400 is free from direct attachment to any portion of the handle assembly 35. For example, the proximal end 404 of the floating tubular bumper 400 may be spaced distally away from the distal end 22 of the distal handle 20. The floating tubular bumper 400 may maintain a fixed axial position during the proximal retraction of the stent retention tube 125, allowing the stent retention tube 125 to move axially relatively to the floating tubular bumper 400. Accordingly, the proximal end 126 of the stent retention tube 125 moves toward the distal end 402 of the floating tubular bumper 400 as the stent retention tube 125 transitions from the distal, delivery position to a proximal, deployment position in which the stent 15 exposed from the stent retention tube 125 and is allowed to radially expand when deployed.

The stent delivery system 40 of FIG. 4A with the stent retention tube 125 in its proximal, deployment position is illustrated in FIG. 4B and FIG. 4C.

In some embodiments, as illustrated in FIG. 4B, the proximal end 126 of the stent retention tube 125 moves closer to and/or abuts the distal end 402 of the floating tubular bumper 400 in its proximal, deployment position. In this configuration, the length of the gap between the stent retention tube 125 and the floating bumper 400 in the delivery configuration (FIG. 4A) must be sufficient in length (e.g., the gap must be at least as great as the length of the stent retention tube 125) such that the stent retention tube 125 may retract a sufficient distance in order to fully uncover the stent 15 before the proximal end 126 of the stent retention tube 125 abuts the distal end 402 of the floating tubular bumper 400.

In some embodiments, the floating tubular bumper 400 may have an outer diameter greater than the inner diameter and/or the outer diameter of the stent retention tube 125. In such an embodiment, the length of the floating tubular bumper 400 must also be such that the stent retention tube 125 may retract a sufficient distance in order to fully uncover the stent 15. In its proximal, deployment position, the stent retention tube 125 will, at maximum, abut the distal end 402 of the floating tubular bumper 400 due to relative size constraints of the floating tubular bumper 400 and the stent retention tube 125, as well as the constraints of the pull thread assembly 100 and the connection points 105.

In other embodiments, the inner diameter of the floating tubular bumper 400, or at least a distal end region thereof, may be greater than the outer diameter of the stent retention tube 125, permitting the proximal end 126 of the stent retention tube 125 to extend int the distal opening of the floating tubular bumper 400 such that the stent retention tube 125 overlaps with the distal end 402 of the floating tubular bumper 400 in its proximal, deployment position. In FIG. 4C, the outer diameter of the stent retention tube 125 is less than the inner diameter of the floating tubular bumper 400, allowing the stent retention tube 125 to slide within the floating tubular bumper 400 as it the stent retention tube 125 transitions into its proximal, deployment position.

In some embodiments, the floating tubular bumper 400 has a stepped or flared configuration, with an enlarged diameter distal end region, wherein the diameter of the floating tubular bumper 400 along its distal end region is greater than the outer diameter of a proximal end region of the floating tubular bumper 400.

FIG. 5A shows a stent delivery system 50 including a stepped floating tubular bumper 400 with the stent retention tube 125 in its distal, delivery position. FIG. 5B shows the same stent delivery system 50 with the stent retention tube 125 in its proximal, delivery position.

In this stepped-bumper configuration, the floating tubular bumper 400 includes an enlarged diameter distal end region 408 extending to the distal end 402 of the floating tubular bumper 400 and a proximal end region 406 extending to the proximal end 404 of the floating tubular bumper 400. The enlarged diameter distal end region 408 may be configured such that the inner diameter at the distal end 402 of the distal end region 408 of floating tubular bumper 400 is sufficiently large as to accommodate the outer diameter of the stent retention tube 125 therein when the stent retention tube 125 is in the distal, delivery configuration surrounding the stent 15. As shown in FIG. 5A, the proximal end 126 of the stent retention tube 125 may be disposed within and overlap with the enlarged diameter distal end region 408 of the floating tubular bumper 400 with the proximal end 126 of the stent retention tube 125 proximal of the distal end 402 of the floating tubular bumper 400 in the distal, delivery configuration surrounding the stent 15. In the illustrated example, the connection points 105 between the pull threads 100a, 100b are located proximate the proximal end 126 of the stent retention tube 125. Accordingly, the distal end region 408 of floating tubular bumper 400 only needs to be sufficiently large to accommodate the outer diameter of the stent retention tube 125 when the stent retention tube 125 is in the proximal, deployment position. In some embodiments, however, the nature of the connection points 105 may dictate that the inner diameter of the distal end region 408 of the floating tubular bumper 400 be sufficiently larger to accommodate the connection points 105. For example, if the connection point 105 is a fitted sleeve with openings for the threads to be knotted to, the distal end region 408 of the floating tubular bumper 400 should have an inner diameter sufficiently large to accommodate for the diameter of the fitted sleeve and the knots securing the pull threads 100a, 100b to the fitted sleeve.

As can be seen in FIG. 5B, in some embodiments, the enlarged diameter distal end region 408 may have a sufficient length (e.g., greater than the length of the stent retention tube 125) such that the distal end region 408 of the floating tubular bumper 400 may extend at least the length needed to accommodate the entire length of the stent retention tube 125 so that the stent 15 can be fully uncovered and self-expand when the stent retention tube 125 is in its proximal, deployment position. Similar to FIG. 4C, the stent retention tube 125 extends int and overlaps with the distal end region 408 of the floating tubular bumper 400 during deployment of the stent 15. In some instances, the stent retention tube 125 may be retracted proximally such that the entire length of the stent retention tube 125 is within the distal end region 408 of the floating tubular bumper 400 in the proximal, deployment position.

In some embodiments, a gap between the distal end 402 of the floating tubular bumper 400 and the proximal end 126 of the stent retention tube 125 may exist when the stent retention tube 125 is in its distal, delivery position. Such a gap may introduce a kink point to the proximal end region of the catheter shaft, which may kink, or otherwise bend, tangle, stretch, twist, sag, drape, or other deform the inner shaft 150 or the pull threads 100a, 100b. If such kink or deformation occurs as the catheter shaft is being advanced into a body lumen, deployment accuracy may be negatively affected, resulting in the stent 15 being released off-target.

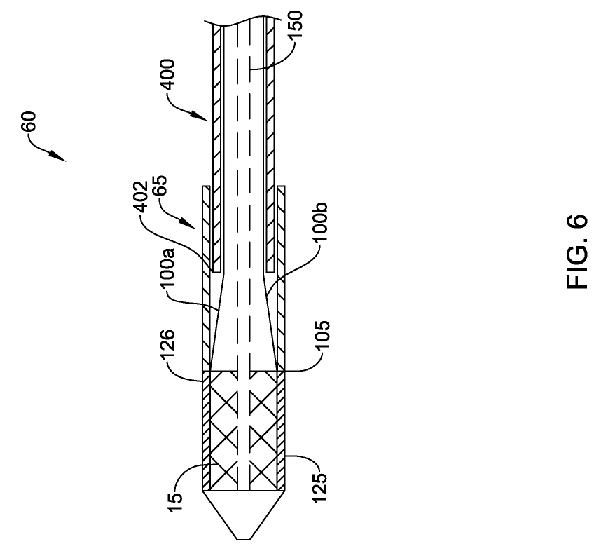

To prevent deformations to the catheter shaft, a transition tube 65, extending proximally from the proximal end 126 of the stent retention tube 125, may be added to stent delivery system 60, as illustrated in FIG. 6. In the illustrated example, the transition tube 65 may have a distal end proximal of and proximate to the proximal end 126 of the stent retention tube 125, while a proximal end of the transition tube 65 ends at a point along the proximal end region 156 of the inner shaft 150. The transition tube 65 may have a length sufficient to cover the gap between the distal end 402 of the floating tubular bumper 400 and the proximal end 126 of the stent retention tube 125 when the stent retention tube 125 is in its distal, delivery position. The transition tube 65 may have any desired length in which the transition tube 65 may extend along the proximal end region 156 of the inner shaft 150 to a location spaced distally from the distal handle 20.

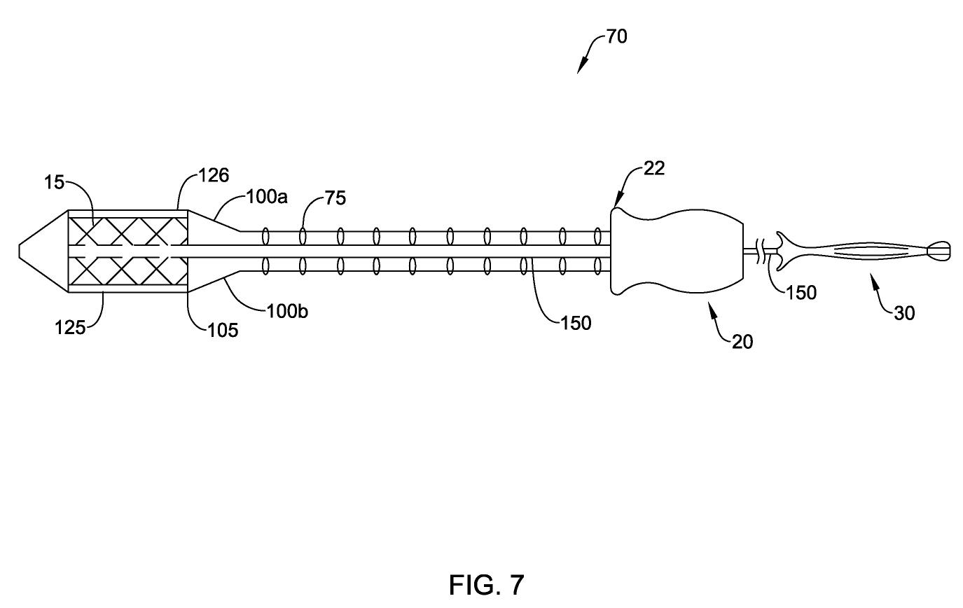

In some examples, the stent delivery system 70 includes a series of guide eyelets 75 positioned longitudinally along the inner shaft 150, as illustrated in FIG. 7. The guide eyelets 75 may be arranged along the exterior of the inner shaft 150. In the illustrated example, the portion of the inner shaft 150 with guide eyelets 75 extends proximally up to the distal end 22 of the distal handle 20. The stent delivery system 70 may include any quantity of guide eyelets 75 evenly or unevenly spaced along the length of the proximal end region of the inner shaft 150. Similar to the floating tubular bumper of other exemplary stent delivery systems 50,60, the guide eyelets 75 serve to keep the pull threads 100a, 100b in close proximity to the exterior of the inner shaft 150 to ensure the integrity of the pull thread assembly and consistent alignment of the pull threads 100a, 100b during deployment, thereby maintaining the reduced friction benefits of the pull thread assembly design of the stent delivery system 70.

The pull threads 100a, 100b may be threaded through the guide eyelets 75 along the proximal end region of the inner shaft 150 between the stent retention tube 125 and the distal handle 20. In other embodiments, the guide eyelets may be made to be longitudinally wider to distribute the localized forces. In other examples, the guide eyelets 75 may be small tubular collars attached to the proximal end region of the inner shaft 150, with the pull threads 100a, 100b extending through the lumen of the tubular collars. Other configurations are also contemplated.

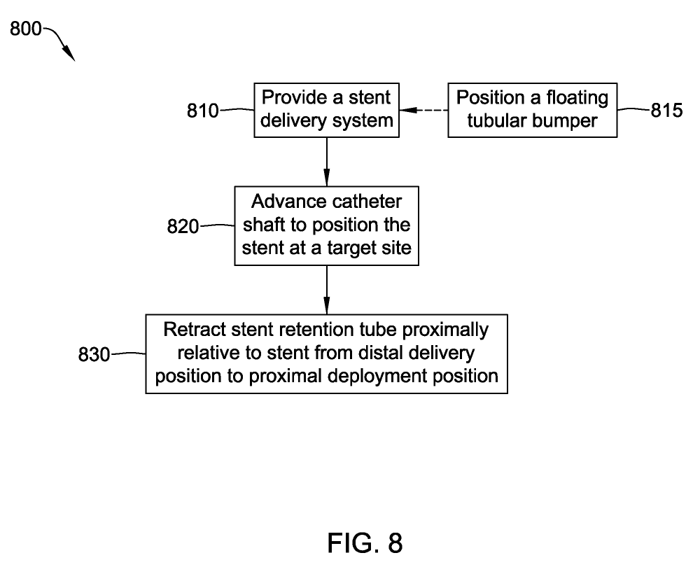

FIG. 8 shows an illustrative method 800 in block flow form. As indicated in block 810, a stent delivery system in accordance with subject matter of the present disclosure is provided. As part of providing 810 the stent delivery system, a floating tubular bumper may be positioned 815 (see FIGS. 4A-C and FIGS. 5A-B) if not already included as a part of the stent delivery system when manufactured. When the procedure is ready to commence, the user may then advance 820 the provided catheter shaft, with the stent retention tube in its distal, delivery position, to position the stent at a target site within a body lumen. To operate the stent delivery system the user actuates the distal handle proximally to proximally retract the pull thread assembly fixedly secured to the stent retention tube. In turn, the pull thread assembly is proximally retracted 830, applying a proximal retraction force to the stent retention tube and transitioning the stent retention tube from its distal, delivery position to its proximal, deployment position. As a result, the stent is exposed and released from the stent retention tube at the target site.

It should be understood that this disclosure is, in many respects, only illustrative. Changes may be made in details, particularly in matters of shape and size, without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A stent delivery system comprising:
a stent;
a catheter shaft comprising:
an inner shaft having a proximal end region and a distal end region;
a stent retention tube having a proximal end and a distal end;
the stent retention tube slidably disposed over the distal end region of the inner shaft;
the stent retention tube configured to hold the stent in a constrained position on the distal end region of the inner shaft;
the stent configured to be deployed from the stent retention tube when the stent retention tube is retracted proximally relative to the stent;
a pull thread assembly comprising one or more pull threads extending along the inner shaft,
wherein the one or more pull threads are fixedly secured to the stent retention tube and extend proximally therefrom along the proximal end region of the inner shaft;
wherein the one or more pull threads are configured to transmit a proximal retraction force to the stent retention tube to retract the stent retention tube proximally relative to the stent.

2. The stent delivery system of claim 1, wherein the catheter shaft further includes a floating tubular bumper circumferentially surrounding the proximal end region of the inner shaft.

3. The stent delivery system of claim 2, wherein the one or more pull threads extend between the inner shaft and the floating tubular bumper.

4. The stent delivery system of claim 3, wherein the stent retention tube is axially movable relative to the floating tubular bumper.

5. The stent delivery system of claim 4, wherein the stent retention tube is axially movable between a distal, delivery position and a proximal, deployment position, wherein in the distal position the stent is restrained within the stent retention tube and in the proximal position the stent is deployed from the stent retention tube.

6. The stent delivery system of claim 5, wherein, in the distal position, a distal end of the floating tubular bumper is spaced proximally away from the proximal end of the stent retention tube, and in the proximal position the proximal end of the stent retention tube is moved toward the distal end of the floating tubular bumper.

7. The stent delivery system of claim 6, wherein in the proximal position, the distal end of the floating tubular bumper abuts the proximal end of the stent retention tube.

8. The stent delivery system of claim 6, wherein in the proximal position, the stent retention tube overlaps the floating tubular bumper.

9. The stent delivery system of claim 5, wherein the floating tubular bumper does not move axially when the stent retention tube is axially moved from the distal position to the proximal position.

10. The stent delivery system of claim 2, further comprising a transition tube extending proximally from the proximal end of the stent retention tube to a distal end region of the floating tubular bumper.

11. The stent delivery system of claim 2, further comprising a handle assembly at the proximal end of the inner shaft, wherein the floating tubular bumper is free from direct attachment to the handle assembly.

12. The stent delivery system of claim 11, wherein the one or more pull threads extend proximal of a proximal end of the floating tubular bumper to the handle assembly.

13. The stent delivery system of claim 1, the pull thread assembly further comprising a plurality of guide eyelets positioned longitudinally along the inner shaft, wherein the one or more pull threads are threaded through the guide eyelets.

14. The stent delivery system of claim 1, wherein the stent retention tube has an outer diameter and the floating tubular bumper has an outer diameter, wherein the outer diameter of the floating tubular bumper is less than the outer diameter of the stent retention tube.

15. The stent delivery system of claim 1, wherein the stent retention tube has an outer diameter and the floating tubular bumper has an inner diameter, wherein the inner diameter of the floating tubular bumper is more than the outer diameter of the stent retention tube.

16. A stent device delivery system comprising:
a stent;
a catheter shaft comprising:
a handle assembly;
an inner shaft extending distally from the handle assembly, the inner shaft having a proximal end region and a distal end region;
a stent retention tube having a proximal end and a distal end;
the stent retention tube slidably disposed over the distal end region of the inner shaft with the proximal end of the stent retention tube spaced apart distally of the handle assembly;
the stent retention tube configured to hold the stent in a constrained position on the distal end region of the stent retention tube;
the stent configured to be deployed from the stent retention tube when the stent retention tube is retracted proximally relative to the stent;
a pull thread assembly comprising one or more pull threads fixedly secured to the stent retention tube, the one or more pull threads extending from a proximal end of the stent retention tube along an exterior of the proximal end region of the inner shaft to the handle assembly,
wherein the one or more pull threads are configured to transmit a proximal retraction force to the stent retention tube to retract the stent retention tube proximally relative to the stent upon actuation of the handle assembly.

17. The stent delivery system of claim 16, wherein the catheter shaft further includes a floating tubular bumper circumferentially surrounding the proximal end region of the inner shaft.

18. The stent delivery system of claim 17, wherein the one or more pull threads extend between the inner shaft and the floating tubular bumper.

19. The stent delivery system of claim 16, wherein the pull thread assembly further comprises a plurality of guide eyelets positioned longitudinally along the proximal end region of the inner shaft, wherein the one or more pull threads are threaded through the guide eyelets.

20. A method of deploying a stent comprising:
advancing a stent delivery system to a treatment site, the stent delivery system comprising:
a self-expanding stent;
a catheter shaft comprising:
an inner shaft having a proximal end region and a distal end region,
a stent retention tube slidably disposed over the inner shaft and axially moveable between a distal, delivery position where the stent is in a constrained position on the distal end region of the inner shaft and a proximal, deployment position where the stent is deployed from the stent retention tube; and
a pull thread assembly comprising one or more pull threads fixedly secured to the stent retention tube and extending proximally therefrom along the proximal end region of the inner shaft; and
retracting the stent retention tube proximally relative to the stent from the distal, delivery position to the proximal, deployment position by transmitting a proximal retraction force through the one or more pull threads, causing the stent to self-expand at the target site.
